(19) United States
(12) Patent Application Publication
(10) Pub. No.: US 2002/0068300 A1
(43) Pub. Date: Jun. 6, 2002

(54) Method for the detection of compounds that modulate the effects of the obese protein

(76) Inventors: Lee James Beeley, Dorking (GB); Richard Anthony Godwin Smith, Horseheath (GB)

Correspondence Address: Glaxosmithkline, Corporate Intellectual Porperty - UW2220, P.O. Box 1539, King of Prussia, PA, 19406-0939, US

(21) Appl. No.: 10/036,956

(22) Filed: Dec. 21, 2001

Related U.S. Application Data
(63) Continuation of application No. 08/952,909, filed on Sep. 8, 1998, now abandoned.
Application No. 08/952,909 is a 371 of international application No. PCT/EP96/02291, filed on May 28, 1996.

(30) Foreign Application Priority Data
May 30, 1995 (GB) 9510857.7
Jun. 8, 1995 (GB) 9511602.6

(51) Int. Cl. C12Q 1/68; C12Q 1/66

(52) U.S. Cl. 435/6; 435/8

(57) ABSTRACT

A method for the detection of a compound that mimics, potentiates or inhibits the physiological effect of the ob-protein, which method comprises: (a) for a compound which mimics the physiological effect of the ob-protein; assessing the effect of the compound upon an ob-protein activated signal transducer and activator of transcription (STAT) NDA response element coupled to a reporter gene; or (b) for a compound which potentiates or inhibits the physiological effect of the ob-protein assessing the effect of the compound upon the response provided by ob-protein upon an ob-protein activated STAT DNA response element coupled to a reporter gene; the response element and the reporter being expressed in an ob-protein responsive cell line; a kit of parts adapted for use in such method and a compound when identified by such method.

The invention relates to a novel method and more particularly to a method for the detection of compounds that mimic, potentiate or inhibit the physiological effects of the ob-protein

The ob-protein (or leptin) is a secreted hormone that acts as signal from adipose tissue to other organs to regulate weight and energy balance (Zhang et. al., Nature, 1994, 372,425). Additional roles for the ob-protein in hematopoietic and reproductive function have been suggested (Cioffi et. al. Nature Medicine, 1996, 2(5), 585). Protein molecules that contain a core composed of four α-helices forming a bundle of up-up-down-down topology comprise of a family of cytokines and growth factors. Proteins of this family cause homo- and hetero-oligermerisation of membrane receptors known to activate kinase cascades resulting in gene transcription. Receptors of the family which are activated by oligermerisation fall into two broad classes; those such as epidermal growth factor, which possess integral tyrosine kinase activity in their intracellular domains (A. Ullrich & J. Schlessinger, Cell, 1990, 61, 203-212), and those such as IL4 and erythropoietin, which lack this activity and mediate their response by way of an associated protein tyrosine kinase (J. N. Ihle et al., TIBS, 1994, 19, 222-227). Both receptor subtypes are activated by cytokines, but the 4-helix bundle proteins activate only the non-integral tyrosine kinase subtype. The non-integral protein tyrosine kinase receptors generally act through a pathway involving Janus kinase (JAK) and their associated signal transducers and activators of transcription (STAT) proteins. On activation STAT proteins bind to DNA response elements thereby controlling gene transcription. Oligonucleotide sequences comprising DNA regulatory elements of the general sequence TT(N)nAA have been identified (Seidel et al., Proc. Nat. Acad. Sci. USA., 1995, 92, 3041) as STAT response elements. These elements bind STAT proteins in response to signaling molecules such as cytokines.

In copending United Kingdom patent application number 9509164.1 we have described our discovery that the ob-protein is characterised by a four helix bundle tertiary structure. We now believe that the ob-protein interacts with a membrane bound receptor that activates a JAK-STAT kinase cascade and hence forms the basis for an assay system for the detection of compounds that mimic, potentiate or inhibit the physiological effects of the ob-protein. Such an assay has utility in selecting compounds for the treatment of weight, energy balance, hematopoietic, fertility and other disorders modulated by the “ob-protein”. The assay is especially useful for selecting compounds for the treatment of those disorders related to obesity, anorexia, cachexia and diabetes.

Accordingly, the invention provides a method for the detection of a compound that mimics, potentiates or inhibits the physiological effect of the ob-protein, which method comprises:

(a) for a compound which mimics the physiological effect of the ob-protein, assessing the effect of the compound upon an ob-protein activated signal transducer and activator of transcription (STAT) DNA response element coupled to a reporter gene; or

(b) for a compound which potentiates or inhibits the physiological effect of the ob-protein, assessing the effect of the compound upon the response provided by ob protein upon an ob-protein activated STAT DNA response element coupled to a reporter gene;

the response element and the reporter being expressed in an ob-protein responsive cell line.

Suitably, the response element is coupled to a promoter gene, preferably a minimal promoter.

A suitable response element is a nucleotide of formula TT(N)ₙAA, where N is any nucleotide and n is 4, 5 or 6.

A favoured response element is selectively activated by the intracellular events mediated the by the ob-protein interacting with its receptor. Such selective response elements can be determined by examining the relative activation of a range of response element-reporter gene constructs when transfected into an ob-responsive cell line by the ob-protein versus other cytokines.

A favoured response element is a nucleotide of formula TT(N)ₙAA, where N is any nucleotide and n is 5.

A further suitable response element is TTCCCGGAA.

A further suitable response element is that region of the promoter of a gene regulated by the ob-protein that is required for STAT interactions. This gene will depend on the particular therapeutic use of the compounds to be selected by the assay.

A suitable reporter gene is firefly luciferase or chloramphenicol acetyltransferase enzyme.

A suitable promoter is a minimal promoter such as the herpes simplex virus thymidine kinase or SV40 promoter.

An example of an “ob-responsive” cell line is a liver or liver hepatoma derived cell line. Liver or liver hepatoma cell lines are available from either the American Type Culture Collection (ATCC) or the Eurpean Collection of Animal Cell Cultures (ECACC). An example of one such cell line is Hep G2 (hepatocellular carcinoma, human) which is available from the ATCC (HB-8065). The Hep G2 cell line is disclosed and claimed by U.S. Pat. No. 4,393,133.

A further example of an “ob-responsive” cell line is the rat-1 fibroblast (Kroder et. al., Exp. Clin. Endocrinol. Diabetes, 1996, 104 (suppl 2), 66). A rat1 fibroblast cell line is available from the ATCC (CRL-2210).

Other responsive cell lines can be identified using a displacement binding assay. Although binding may not be to a functional long form of the receptor, which is the form that transmits a signal to the cytoplasm. Identification of a functional long form of the receptor may be by PCR or Northern blot analysis (eg. Human ob-receptor: Tartaglia et al., Cell, 1995, 83, 1263). Ultimately responsive cells are detected by monitoring cellular events in the presence of varying concentrations of leptin. Potential methods for identifying candidate cell lines or monitoring these cellular events include the following:

1. Microphysiometer: This method detects small changes in pH resulting frombiochemical changes in the cell. Ob-protein responsive cells upon stimulation may undergo biochemical changes that cause a small change in the extracellular acidification rate which can be detected by a silicon microphysiometer. The microphysiometer biosensor methodology has been reviewed by McConnell, Science, 1992, 257, 1906.

2. Electrophoretic mobility shift assay (EMSA): Nuclear extracts from cells after treatment with ob-protein are mixed with radiolabeled oligonucleotides containing a promiscuous or specific STAT response element DNA sequence. Extracts from cells that respond to the ob-protein may cause a gel shift of the oligonucleotide for the STAT response element. References: Book “Recombinant DNA”, 2nd Edition, Watson et al., 1992, Page 158; Lamb et al., Blood, 1994, 83, 2063;

3. Measurement of protein phosphorylation assay: The coupling of receptor activation to the final response through tyrosine phosphorylation of intracellular proteins may be assayed by the use of antibodies recognising phosphorylated tyrosines. More specifically since the leptin receptor may stimulate tyrosine phosphorylation of the JAK/STAT pathway this method provides a method of detecting leptin response cell lines. Specific JAK/STAT antibodies may be used alongside antibodies for tyrosine phosphorylation to detect leptin activation in a leptin responsive cell line. Inhibition as well as stimulation of protein phosphorylation may occur. In particular, inhibition by the ob-protein of insulin stimulated phosphorylation of the insulin receptor and insulin receptor substrate-1 has been shown in rat-1 fibroblasts overexpressing insulin receptors (Kroder et. al 1996, Exp. Clin. Endocrinol. Diabetes, 104, suppl 2, p66)

4. Displacement binding: After incubation of cell lines with radiolabelled leptin, for example [¹²⁵I]-leptin, the non-specific binding versus specific binding of leptin can studied by the addition of unlabelled leptin. A high specific to non-specific ratio binding suggests that the cell line may contain the leptin receptor.

5. Detection of the protein for a functional form, preferably a functional long form, of the ob-receptor by use of selective antibodies.

6. Detection of mRNA for a functional form, preferably a functional long form, of the ob-receptor by Northern, RT-PCR or “slot blot” analysis.

Cell lines known to be involved in controlling aspects of the particular disease state for which compounds are being sought are preferred.

Cells lines derived from liver, brain, or pancreatic tissue and fibroblasts are particularly useful for “ob-responsive” cells for the assaying of compounds directed at obesity and diabetes. Certain areas of the brain are the focus of weight controlling and energy balance regulating effects of the ob-protein. The liver controls many metabolic processes that modulate lipid and glucose levels. Cells derived from particular regions of these organs containing the appropriate endogenous JAKs, STAT proteins and other intracellular proteins which are required for mediating the effects of the leptin are preferred.

The response element, the reporter, and preferably the promoter, are suitably incorporated into a vector capable of transfecting the ob-responsive cell line.

Suitable vectors are commercially available vectors, such as pGL2-basic luciferase vector (Promega).

A suitable configuration of the vector is the STAT DNA response element upstream of a promoter and a reporter gene. A more suitable configuration of the vector is the STAT DNA response element in multiple tandem repeats (2-10) upstream of a thymidine kinase promoter and a luciferase reporter gene

Vectors are constructed containing a reporter gene for example firefly luciferase or chloramphenicol acetyltransferase enzyme linked to a minimal promoter for example the herpes simplex virus thymidine kinase or SV40 promoter. The DNA fragments for the STAT response element are inserted into the vector using appropriate restriction enzyme sites upstream of the minimal promoter.

The response element, the reporter and the promoter, as required, are incorporated into the vector using conventional expression techniques, for example the DNA fragments for the response element may be inserted into the vector using appropriate restriction enzyme sites upstream of the minimal promoter.

STAT response element-luciferase enzyme reporter systems can be constructed as described by Lamb et al., Blood, 1994, 8, 2063 and Seidel et al., Proc. Nat. Acad. Sci. USA., 1995, 92, 3041.

Ob-responsive cells are transfected with the STAT response element-minimal promoter-luciferase reporter constructs using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Potentiation or antagonist activity can be assayed by pre- or co-addition of an appropriate concentration of ob-protein to the compound under evaluation and measuring the potentiation or reduction in luciferase response relative to that of ob-protein alone. Standard methods exist for assaying luciferase enzyme activity for example Ow et al., Science, 1986, 234, 856 and de Wet et al., Mol. Cell Biol., 1987, 7, 725. as well as several commercial kits.

Stable cell lines can be generated by transfecting an “ob-responsive” cell line with the reporter construct and a selectable marker. Selectable markers are routinely used to generate stable cell lines as described in Recombinant DNA, 2nd edition, J. D. Watson et. al., 1992, page 216. These stably transfected cell lines can be used to generate high throughput assays for compounds that mimic, potentiate or block the physiological effects of the ob-protein.

The invention also extends to a compound that mimics, potentiates or inhibits the physiological effect of the ob-protein, when identified by the method disclosed herein.

The invention also extends to a kit of parts adapted for use in the method disclosed herein.

When used herein ‘a compound which mimics the physiological effects of the ob-protein’ refers to a compound which is capable of acting in the absence of the ob-protein to either stimulate the ob-protein receptor to provide substantially the same physiological effect as the ob protein or to activate a response down stream of this receptor (post-receptor).

When used herein ‘a compound that potentiates the physiological effect of the ob-protein’ refers to a compound which enhances the potency and/or maximal physiological effect of the ob-protein.

When used herein ‘a compound that inhibits the physiological effect of the ob-protein’ refers to a compound which reduces or substantially blocks the physiological effect of the ob protein.

The following example illustrates the invention

### EXAMPLE

### General Procedure

Ob-responsive cells are transfected with a reporter plasmid containing a STAT response element, in multiple tandem copies upstream of a minimal promoter for example herpes simplex thymidine kinase and a luciferase gene reporter constructusing standard methodology for example the calcium phosphate method (Graham and Van Der Eb, Virology, 1973, 52, 456). To correct for differences in taansfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist activity can be assayed by pre- or co-addition of an appropriate concentration of ob-protein to the compound under evaluation and measuring the reduction in luciferase response relative to that of ob-protein alone. Standard methods exist for assaying luciferase enzyme activity for example Ow et al., Science, 1986, 234, 856 and de Wet et al., 1987, 7, 725. as well as several commercial kits.

### Example

A liver hepatoma derived cell line is transfected with a reporter plasmid, pGL2-basic luciferase vector (promega) containing an insert of an oligonucleotide corresponding to a four fold tandem repeat of the STAT response element, TTCCCGGAA, upstream of the minimal promoter for herpes simplex thymidine kinase (−35 to +10 ) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist activity can be assayed by pre- or co-addition of an appropriate concentration of ob-protein to the compound under evaluation and measuring the reduction in luciferase response relative to that of ob-protein alone. Standard methods exist for assaying luciferase enzyme activity for example Ow et al., Science, 1986, 234, 856 and de Wet et al., Mol. Cell Biol., 1987, 7,725. as well as several commercial kits.

1. A method for the detection of a compound that mimics, potentiates or inhibits the physiological effect of the ob-protein, which method comprises:
(a) for a compound which mimics the physiological effect of the ob-protein, assessing the effect of the compound upon an ob-protein activated signal transducer and activator of transcription (STAT) DNA response element coupled to a reporter gene; or
(b) for a compound which potentiates or inhibits the physiological effect of the ob-protein, assessing the effect of the compound upon the response provided by ob protein upon an ob-protein activated STAT DNA response element coupled to a reporter gene;
the response element and the reporter being expressed in an ob-protein responsive cell line.

2. A method according to claim 1, wherein the response element is coupled to a promoter gene, preferably a minimal promoter.

3. A method according to claim 2, wherein the response element is a nucleotide of formula TI(N)ₙAA, where N is any nucleotide and n is 4, 5 or 6, preferably 5.

4. A method according to claim 2, wherein the response element is a nucleotide of formula TTCCCGGAA.

5. A method according to claim 1, wherein the reporter gene is firefly luciferase or chloramphenicol acetyltransferase enzyme.

6. A method according to claim 1, wherein the promoter is the herpes simplex virus thymidine kinase or SV40 promoter.

7. A method according to claim 1, wherein the ob-responsive cell line is a liver or liver hepatoma derived cell line.

8. A method according to claim 1, wherein the response element, the reporter, and the promoter, are incorporated into a vector capable of transfecting the ob-responsive cell line.

9. A method according to claim 8, wherein the vectors pGL2-basic luciferase vector (Promega).

10. A method according to claim 8 or claim 9, wherein the configuration of the vector is such that the STAT DNA response element is upstream of the promoter and reporter gene.

11. A kit of parts adapted for use in the method for the detection of a compound that mimics, potentiates or inhibits the physiological effect of the ob-protein, which method comprises:
(a) for a compound which mimics the physiological effect of the ob-protein, assessing the effect of the compound upon an ob-protein activated signal transducer and activator of transcription (STAT) DNA response element coupled to a reporter gene; or
(b) for a compound which potentiates or inhibits the physiological effect of the ob-protein, assessing the effect of the compound upon the response provided by ob protein upon an ob-protein activated STAT DNA response element coupled to a reporter gene;
the response element and the reporter being expressed in an ob-protein responsive cell line.
